SEQUENCE LISTING (2 sequences)

SEQ ID NO: 1 - DNA, 21 bp, Artificial Sequence
  GREB1-specific sgRNA target sequence
gctgacgggacgacacatctg

SEQ ID NO: 2 - DNA, 21 bp, Artificial Sequence
  ESR1-specific sgRNA target sequence
gtctccagcagcaggtcatag

(19) United States
(12) Patent Application Publication
(10) Pub. No.: US 2021/0172934 A1
(43) Pub. Date: Jun. 10, 2021

(54) SINGLE CELL CLONING APPROACHES FOR BIOLOGICAL STUDIES

(71) Applicant: The Brigham and Women`s Hospital, Inc., Boston, MA (US)

(72) Inventors: Sandra S. McAllister, Boston, MA (US); Jessica F. Olive, Worcester, MA (US)

(21) Appl. No.: 17/119,245

(22) Filed: Dec. 11, 2020

Related U.S. Application Data
(63) Continuation of application No. PCT/US2019/036554, filed on Jun. 11, 2019.
(60) Provisional application No. 62/683,225, filed on Jun. 11, 2018.

(51) Int. Cl. G01N 33/50; C12N 15/11; C12N 9/22

(52) U.S. Cl. CPC G01N 33/5011; C12N 15/11; C12N 2800/80; C12N 2310/20; C12N 9/22

(57) ABSTRACT

Clonally derived barcoded cell populations and methods of generating the same, as well as methods of use thereof, e.g., to evaluate heterogeneity of a starting population of cells.

### CLAIM OF PRIORITY

This application is a continuation of PCT/US2019/036554, filed on Jun. 11, 2019, which claims the benefit of U.S. Provisional Application Ser. No. 62/683,225, filed on Jun. 11, 2018. The entire contents of the foregoing are incorporated herein by reference.

### FEDERALLY SPONSORED RESEARCH OR DEVELOPMENT

This invention was made with Government support under Grant No. CA166284 awarded by the National Institutes of Health and Grant No. W81XWH-14-1-0191 awarded by the Department of Defense. The Government has certain rights in the invention.

### TECHNICAL FIELD

Described are clonally derived barcoded cell populations and methods of generating the same, as well as methods of use thereof, e.g., to evaluate heterogeneity of a starting population of cells.

### BACKGROUND

The relevance and pervasiveness of genetic and functional heterogeneity within most cancer types has become particularly appreciated over the past decade [8-10]. It is now known that even supposedly clonal cancer cell lines are composed of subpopulations with widely differing phenotypes and functional characteristics [11-13]. Genetic and phenotypic heterogeneity has also been observed in other disease models, including bacterial antibiotic resistance and in the evolution of antiviral resistance [14-17].

CRISPR/Cas9 is a useful tool that has expanded our ability to define the role of particular factors in biological processes, including cancer biology [1, 2]. Oftentimes, studies employ the CRISPR/Cas9 system to generate loss- or gain-of-function mutations in a gene of interest and then look for a corresponding phenotypic change, indicating whether or not the targeted gene is necessary and/or sufficient for a particular behavior. Widely used protocols that employ CRISPR/Cas9 to generate genetically modified cell lines often require a subcloning and/or selection step in order to isolate a particular subpopulation in which the gene of interest was efficiently edited [3-7]. In order to correctly define the role that particular factors play, for example in cancer models, it is essential to use appropriately matched controls to compare to the edited subclone(s); however, such comparisons can be complicated by the widespread heterogeneity present in tumors and cancer cell lines derived from them.

### SUMMARY

Gene editing protocols often require the use of a subcloning step to isolate successfully edited cells, the behavior of which is then compared to the aggregate parental population and/or other non-edited subclones. The results herein demonstrate that the inherent functional heterogeneity present in many cell lines can render these populations inappropriate controls, resulting in erroneous interpretations of experimental findings. The present protocol incorporates a single-cell cloning step prior to gene editing, allowing for the generation of appropriately matched, functionally equivalent control and edited cell lines. The results demonstrate that heterogeneity should be considered during experimental design when utilizing gene editing protocols and provide a solution to account for it.

Thus provided herein are methods for creating a plurality of barcoded clonal populations (BCPs). The methods include (a) providing an initial heterogeneous population of cells; (b) dividing the initial heterogeneous population of cells into separate cultures, each culture comprising a single cell from the initial heterogeneous population; (c) maintaining the single cells in culture to provide a plurality of stable single cell-derived monoclonal populations; and (d) introducing individual identifying nucleic acid sequences into each cell of the plurality of stable single cell-derived monoclonal populations; to thereby create a plurality of barcoded clonal populations (BCPs).

In some embodiments, the methods include (e) mixing equal numbers of each BCP to create a barcoded polyclonal population of cells (BPP). In some embodiments, the methods include exposing the BPP to a test condition. In some embodiments, the methods include determining one or both of identity and relative abundance of each BCP in the BPP, e.g., using a method comprising PCR, a hybridization assay, or next-generation sequencing.

In some embodiments, the initial heterogeneous population of cells comprises cells from cancer cell lines (e.g., from a single cancer cell line) or patient-derived cells, e.g., from a single patient, optionally including cells from normal tissues, e.g., affected and/or normal cells.

In some embodiments, the identifying nucleic acid sequences comprise unique sequences of 10-40 nucleotides.

In some embodiments, the identifying nucleic acid sequences comprise unique sequences of 20-30 nucleotides, e.g., 24 nucleotides.

In some embodiments, the identifying nucleic acid sequences are flanked by uniform sequences comprising PCR primer binding sites. The sites allow for PCR amplification of the identifying nucleic acid sequences from genomic DNA preparations.

In some embodiments, the identifying nucleic acid sequences are integrated into the genomes of the cells of the plurality of stable single cell-derived monoclonal populations.

In some embodiments, the identifying nucleic acid sequences are introduced into the cells of the plurality of stable single cell-derived monoclonal populations using a viral vector. In some embodiments, the viral vectors are lentiviral vectors.

Also provided herein are methods for selecting a therapy for a subject who has a cancer. The methods comprising: creating a plurality of barcoded clonal populations (BCPs) by a method comprising (a) providing an initial heterogeneous population of cells from the cancer in the subject; (b) dividing the initial heterogeneous population of cells into separate cultures, each culture comprising a single cell from the initial heterogeneous population; (c) maintaining the single cells in culture to provide a plurality of stable single cell-derived monoclonal populations; and (d) introducing individual identifying nucleic acid sequences into each cell of the plurality of stable single cell-derived monoclonal populations; to thereby create a plurality of barcoded clonal populations (BCPs); (e) mixing equal numbers of each BCP to create a barcoded polyclonal population of cells (BPP); (f) exposing the BPP to a candidate therapeutic compound; and determining one or both of identity and relative abundance of each BCP in the BPP.

In some embodiments, identity and/or relative abundance of each BCP is determined using a method comprising PCR, a hybridization assay, or next-generation sequencing.

In some embodiments, the initial heterogeneous population of cells comprises cells from cancer cell lines (e.g., from a single cancer cell line) or patient-derived cells, e.g., from a single patient, optionally including cells from normal tissues, e.g., affected and/or normal cells.

In some embodiments, the identifying nucleic acid sequences comprise unique sequences of 10-40 nucleotides.

In some embodiments, the identifying nucleic acid sequences comprise unique sequences of 20-30 nucleotides, e.g., 24 nucleotides.

In some embodiments, the identifying nucleic acid sequences are flanked by uniform sequences comprising PCR primer binding sites. The sites allow for PCR amplification of the identifying nucleic acid sequences from genomic DNA preparations.

In some embodiments, the identifying nucleic acid sequences are integrated into the genomes of the cells of the plurality of stable single cell-derived monoclonal populations.

In some embodiments, the identifying nucleic acid sequences are introduced into the cells of the plurality of stable single cell-derived monoclonal populations using a viral vector. In some embodiments, the viral vectors are lentiviral vectors. Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1G. Phenotypic and functional heterogeneity of McNeuA and Met-1 breast cancer cells. (1A) Concentration of murine OPN (mOPN; ng/ml per 10⁶ cells) in 24-hr conditioned medium of McNeuA and Met-1 murine mammary carcinoma cells represented as mean±SD. There was no detectable mOPN in the control cell-free medium (DMEM) (2 technical replicates per group). (1B) Incidence of tumor formation following injection of indicated numbers of McNeuA or Met-1 cells into cohorts of FVB mice. (1C) Plasma mOPN concentration (ng/ml) in indicated cohorts of mice at experimental end points of 84 days (McNeuA) and 30 days (Met-1). For McNeuA tumor-bearing mice, blue data points represent 10,000 cells injected, red data points represent 100,000 cells injected; n=6-7 for McNeuA cohorts; n=5-8 for Met-1 cohorts. Error bars represent SD; statistical significance evaluated using unpaired, two-tailed Student's t-test. (1D) Representative images of immunohistochemical staining for murine E-cadherin (red) on recovered McNeuA and Met-1 tumors. Cell nuclei were counterstained with hematoxylin (blue). Scale bars=100 um. (1E) Average radiance (log₁₀) per mouse (n=5) as measured by bioluminescence imaging over 21-day time course following intravenous injection of 10⁶ Met-1 tumor cells into FVB mice (left graph). Fold-change (log₂) in pulmonary metastatic burden per mouse (right graph). Representative of 2 independent experiments. (1F) Response of orthotopic Met-1 GFP/Luc tumors to single dose combination doxorubicin (5 mg/kg), paclitaxel (10 mg/kg) and cyclophosphamide (120 mg/kg) (AC-T), n=5-8 tumors/group. Ordinate represents time (days) following treatment. Error bars represent SEM; two-way ANOVA Sidak's multiple comparisons test; *p<0.01. Representative of 3 independent experiments. (1G) Growth kinetics of individual orthotopic Met-1 Luc/GFP tumors in mice injected with 2.5×10⁵ tumor cells at the experiment initiation, subsequently receiving 4 biweekly AC-T doses (red arrows). Numbers and colors represent individual mice.

FIGS. 2A-2E. Phenotypic heterogeneity of McNeuA and Met-1 subclonal populations. (2A) Schematic of subclone derivation from breast cancer cell lines. (2B,2C) Phase contrast images of representative McNeuA (2B) and Met-1 (2C) subclones to demonstrate morphologic variability. Scale bars=100 um. (2D,2E) Concentration of murine osteopontin (mOPN; ng/ml per 10⁶ cells) in 24-hr conditioned media from McNeuA (MC) sublcones (2D) and Met-1 (MT) subclones (2E).

FIGS. 3A-3F. McNeuA and Met-1 subclonal populations are functionally heterogeneous in tumor incidence, latency and growth kinetics. (3A,3B) Primary tumor incidence of indicated McNeuA (10⁵ or 10⁶ cells; A) and Met-1 (2.5×10⁴ or 2.5×10⁵ cells; B) clonal populations that were injected orthopically into FVB mice. (3C,3D) Tumor growth kinetics of indicated McNeuA clones that were orthotopically injected into FVB mice at 10⁵ (3C) or 10⁶ (3D) cells. Error bars represent SD; statistical significance evaluated using 2way-ANOVA. (3E,3F) Tumor growth kinetics of indicated Met-1 clones that were orthotopically injected into FVB mice at 2.5×10⁴ (3E) or 2.5×10⁵ (3F) cells. Error bars represent SD; statistical significance evaluated using 2way-ANOVA.

FIGS. 4A-4F. Generation of appropriately matched wild-type and OPN knockout cell lines using CRISPR-Cas9 mediated gene editing. (4A) Schematic of traditional and modified CRISPR/Cas9 based gene editing protocols. (4B) Schematic diagram of sgRNA targeting the spp1 gene loci (SEQ ID NO:4). Protospacer sequence is highlighted in red. Protospacer adjacent motif (PAM) sequences are presented in green. (4C) Recovery rates, gene editing efficiency, and rate of homozygous targeting of the OPN gene in indicated subclones. (4D) Western blot for OPN protein in MC-22 WT and edited clones (P16, P23, and P38) cultured in the presence or absence of brefeldin A (BFA). Expected multiple Osteopontin isoforms were detected between 37-50 kD. A non-specific band was detected in each sample, indicated by an asterisk. (4E) Concentration of murine osteopontin (mOPN) in 24-hr conditioned media from MC-50 WT and edited clones (MC-50-KO1 and MC-50-KO2). mOPN levels were normalized to final cell count. Osteopontin was undetected (ND) in conditioned media collected from both edited subclones. (4F) Immunofluorescence cytochemical staining for mOPN (red) in MT-2 WT and a validated MT-2 OPN-KO clone. Nuclei are counterstained with hematoxylin (blue). Scale=100 μm.

FIGS. 5A-5D. OPN depletion does not affect primary tumor formation in murine models of HER2⁺ and ER⁻ breast cancer. (5A-5C) FVB mice were orthotopically injected with 10⁵ MC-22 (5A), 10⁵ MC-50 (5B), or 2.5×10⁴ MT-2 (5C) cells. Growth kinetics (mm³) of orthotopic tumors of WT (blue lines) and validated OPN-KO clones (red lines). Mass of primary tumors from WT (blue) or OPN-KO (red) cohorts at experimental end points. No statistically significant differences determined by 2way ANOVA (tumor growth kinetics) or unpaired, two-tailed Students' t-test (tumor mass) statistical analyses. Circulating plasma murine osteopontin (mOPN) levels from cancer-free (green) or tumor bearing mice from the MC-22, MC-50, or MT-2 WT (blue) or OPN-KO (red) cohorts (One-way ANOVA: *** p=0.0003, **** p<0.0001). Error bars represent SD. (5D) Representative immunohistochemical staining for mOPN (red) in tumors derived from MC-22, MC-50 and MT-2 WT and validated OPN-KO cell lines. Cell nuclei counterstained with hematoxylin (blue). Scale bar=50 μm.

FIGS. 6A-6F. Matched wild type and knockout OPN cell lines can be used for pre-clinical metastasis studies. (6A) Experimental schema for metastasis assay. (6B) Representative in vivo bioluminescent images of mice injected with MT-2 WT or MT-2 OPN KO after 7d and 21d. (6C) Average bioluminescent signal (radiance, log 10) from mice with MT-2 WT (blue) or MT-2 OPN KO (red) at indicated time points. (Mann-Whitney: ** p=0.0085). (6D) Representative hematoxylin & eosin staining of lungs from mice that received tail vein injections of MT-2 WT or MT-2 OPN KO cells. An example of a multifocal metastasis is marked with a blue arrow and an example of a single focus metastasis is marked with a red arrow. Scale=100 (6E)_Quantification of total metastases in MT-2 WT (blue) and MT-2 OPN KO (red) cohorts (WT n=21, KO n=30; Mann-Whitney, p=0.0466). Error bars represent SD. (6F) Quantification of multifocal metastases in MT-2 WT (blue) and MT-2 OPN KO (red) cohorts (WT n=21, KO n=30; Mann-Whitney, p=0.0185). Error bars represent SD.

FIGS. 7A-7C. MT-2 OPN-KO derived tumors exhibit enhanced chemosensitivity in vivo.

(7A) Experimental schema. 2.5×10⁴ MT-2 WT or OPN-KO tumor cells were injected into the mammary fat pads of 6-8-week-old female FVB mice. A single dose of AC-T was initiated at 14 days, when tumors reached ˜60-80 mm³ in volume, and tumor growth was monitored periodically until the end point of 44 days. Error bars represent SD. (7B) Tumor growth kinetics for MT-2 WT vehicle (blue; n=5) and AC-T treated (green; n=4) and MT-2 OPN-KO vehicle (red; n=3) and AC-T treated mice (purple; n=2). Representative of 3 biological repetitions. Error bars represent SD. (7C) Endpoint tumor mass for MT-2 WT and MT-2 OPN-KO AC-T treated mice from 2 separate experiments (Mann-Whitney, p=0.0037; endpoint tumor mass was not measured during the first of the three experimental repetitions). Data points from individual repetitions are represented with different colors. Error bars represent SD.

### DETAILED DESCRIPTION

Due to the inherent functional heterogeneity observed in most cancer cell lines, subcloning and selection steps employed in genetic editing protocols can render the parental population an inappropriate control, as its behavior may differ from that of the selected subclonal population prior to gene editing. For example, if the aim of a study is to evaluate whether a particular gene product (protein) is relevant for primary tumor formation, it is common practice to compare the tumorigenicity of a knockout cell line with that of the parental cell line. However, if the selected subclonal population has an inherently different tumorigenic potential than the bulk parental population, it would be possible to incorrectly conclude that the knockdown of the gene of interest was responsible for any functional differences that are observed in any given biological assay.

The ability to genetically edit a cell line to either suppress, knockdown, induce, overexpress, knock-in, or mutate a protein of interest provides an indispensible tool for biological research. However, our work demonstrates that studies designed to test necessity or sufficiency of genes/gene products without choosing appropriately matched unedited controls run the risk of detecting false positive or false negative results due to inherent phenotypic differences in subclonal cellular populations that result from heterogeneity. Our alternative approach to generate subclones and screen for desired phenotypes prior to genetic manipulation provides one solution to this problem.

As demonstrated herein, our approach worked well for hypothesis-testing experimentation, when biological phenotypes to be tested are defined. Another benefit to our modified approach is that characterization of subclone phenotypes may enable one to select a range of biological properties that could be tested. Moreover, this approach enables discovery of novel properties for which mechanistic insight could be obtained in a straightforward manner. For example, one of our subclones (MC-22 KO) stimulated elevated host plasma OPN while another clone (MC-50 KO) did not, thereby enabling one to compare properties (e.g., gene expression) of related clones to yield mechanistic insights. Our approach ensures that the real function of a specific protein of interest is uncovered during experimentation.

The present disclosure provides methods of evaluating variation across cell line strains, which should be considered in experimental design and data interpretation.

The barcoding approaches described herein can be used to track and study individual subclonal populations within a heterogeneous populations of cells or tissues. Traditional cell tagging approaches currently do not enable one to enumerate cells at the end point of a study or know anything about their identity or the ability to isolate them for further study. Therefore, the present inventors developed a molecular barcoding approach that enables the analysis of intratumoral subclonal composition, tracking of cells over time, and retrieval of barcoded cells for further study.

Importantly, the present approach is different from others that have been reported in that we generate single cell subclones prior to introducing the barcode tags. Other reported approaches infect heterogeneous parental populations of cells with an entire library of barcodes at low MOI, without the ability to identify which cells are tagged with which barcode. Hence, one advantage of the present approach is that by introducing single barcodes into monoclonal populations and then generating the pooled barcoded polyclonal population rather than infecting the bulk parental population with a library of barcodes, we gain the ability to retroactively characterize barcoded monoclonal populations in any given experiment. This approach also allows us to be confident that the same barcode is not unwittingly introduced into multiple unique clonal populations, thus confounding subsequent analyses.

The general methods are as follows. First stable single cell-derived monoclonal populations (CPs) are generated from heterogeneous populations of cells, e.g., cultured cells (e.g., cancer cell lines, e.g., any of the NCI-60 cancer cell lines, see, e.g., dtp.cancer.gov/discovery development/nci-60/cell list.htm) or patient-derived cells, e.g., affected and/or normal cells; affected cells are cells that are affected by a disease, e.g., tumor cells. Methods for obtaining and culturing the cells are known in the art. For example, cells are separated (e.g., by dilution or cell sorting) into individual cells that are placed into individual culture environments, e.g., individual vials or wells of a culture dish. Where the cells are obtained from a patient tumor or other tissue, they can first be dissociated, e.g., enzymatically, chemically, or mechanically. The individual cells are then maintained in culture to produce individual clonal populations. Any number of individual clonal populations can be produced, e.g., 10, 100, 1000, 10⁴, 10⁵, 10⁶, or more.

Each individual clonal population is then tagged with a unique molecular “barcode” sequence (also referred to herein as individual identifying nucleic acid sequence), e.g., using a viral vector, e.g., recombinant retroviruses, adenovirus, adeno-associated virus, alphavirus, and lentivirus vectors (Yu, et al. Nat Biotech 2016) to create barcoded clonal populations (BCPs). The individual identifying nucleic acid sequences preferably are integrated into the genome of the cells. In preferred embodiments, upon integration, each individual identifying nucleic acid sequence introduces a unique heritable DNA barcode tag of 10-50 base pairs, 20-30 base pairs, e.g., 24-base pairs, into each cell clone genome; these individual identifying nucleic acid sequences can be used to precisely follow the progeny of each cell over time. Each individual identifying nucleic acid is flanked by uniform sequences that are common to all of the cells and allow for PCR amplification of the individual identifying nucleic acid sequences from genomic DNA preparations made from the cells.

In some embodiments, substantially (i.e., within about plus or minus 10%, given difficulties in exactly determining numbers of cells) equal numbers of each BCP, or known ratios of each BCP, are then mixed together to create a barcoded polyclonal population of cells (BPP). The BPP can be exposed to a number of conditions. Then the identity and relative abundance of each clonal population (BCP) within a polyclonal mixture of cells (BPP), e.g., optionally including tumor and non-tumor stromal cells, is determined, e.g., using a Luminex-based PRISM detection (Yu, et al., Nat Biotech, 2016) or next-generation sequencing. These methods allow the identification and quantification of the representation of each individual barcode in a given tissue sample.

In some embodiments, one caveat of our approach is that isolating particular subclonal populations removes the inherent heterogeneity of a cell line, which could have important biological consequences. This is particularly relevant in circumstances in which the biology is not well understood. If heterogeneity is desirable, then one could employ a clonal pooling approach, thus ensuring that a given experiment is both properly controlled and that the heterogeneous nature of the parental cell line is not lost.

It has been reported that functional heterogeneity can arise even within a ‘clonal’ cellular population as a result of cell plasticity or epigenetic alteration [13]. Hence, although we did not test clonal plasticity in our system, it is reasonable to hypothesize that a high degree of cellular plasticity could cause differences between matched control and edited populations that may not be due to the target gene. Limiting the in vitro passage of the cell lines to minimize chances for additional selection and monitoring for unexpected functional changes in control cell lines may help to prevent this added complication.

While other gene editing, barcoding, and other cell manipulation technologies exist, the present methods derive individual clonal populations of cells from a parental cell line or tissue prior to any type of modulation so that individual clonally related cells can be tracked and studied in any given experiment. The barcode detection system can also be optimized for applicability with typical DNA sequencing technologies.

Provided herein are collections of single cell clonal populations derived from cell lines or tissues; collections of single cell clonal populations tagged with unique molecular barcodes; and collections of mixed populations of cells comprised of uniquely barcoded clonal populations.

The presence of an individual identifying nucleic acid sequences as described herein can be evaluated using methods known in the art, e.g., using polymerase chain reaction (PCR), reverse transcriptase polymerase chain reaction (RT-PCR), quantitative or semi-quantitative real-time RT-PCR, multiplex PCR, digital PCR, e.g., BEAMing ((Beads, Emulsion, Amplification, Magnetics), Diehl (2006) Nat Methods 3:551-559); various types of nucleic acid sequencing (Sanger, pyrosequencing, NextGeneration Sequencing); multiplexed gene analysis methods, e.g., oligo hybridization assays including DNA microarrays; hybridization based digital barcode quantification assays such as the nCounter® System (NanoString Technologies, Inc., Seattle, Wash.; Kulkarni, Curr Protoc Mol Biol. 2011 April; Chapter 25:Unit25B.10) and hybridization assays, e.g., utilizing branched DNA signal amplification such as the QuantiGene 2.0 Single Plex and Multiplex Assays (Affymetrix, Inc., Santa Clara, Calif.; see, e.g., Linton et a., J Mol Diagn. 2012 May-June; 14(3):223-32); SAGE; MLPA; or luminex/XMAP. See, e.g., WO2012/048113, which is incorporated herein by reference in its entirety.

The methods described herein can include exposing the barcoded polyclonal population of cells (BPPs) to test conditions, e.g., the presence or absence of one or more environmental factors (e.g., temperature, light, atmosphere (e.g., levels of oxygen or nitrogen) or test compounds (e.g., polypeptides, polynucleotides, inorganic or organic large or small molecule test compounds) to determine whether different BCPs within the BPP react differently to the test conditions.

As used herein, “small molecules” refers to small organic or inorganic molecules of molecular weight below about 3,000 Daltons. In general, small molecules useful for the invention have a molecular weight of less than 3,000 Daltons (Da). The test compounds can be, e.g., natural products or members of a combinatorial chemistry library.

The methods can include comparing genetic, genomic, epigenomic, transcriptomic, proteomic, and other profiles across and within the BCPs, e.g., preferably before being combined in a BPP, to determine heterogeneity of a starting population of cells. The methods can alternatively or in addition include determining effects on viability, proliferation, motility, cell cycle, or other cellular characteristics. In some embodiments, one or more characteristics of each BCP is determined before they are mixed together to form a BPP, e.g., genetic, genomic, epigenomic, transcriptomic, proteomic, or other profiles, or viability, proliferation, motility, cell cycle, or other cellular characteristics can be determined; such characteristics can be determined using methods known in the art. For example, the presence of therapy-resistant BCPs can be identified. In some embodiments, therapy-resistant cells are present naturally in the starting sample, and make up some proportion of the BCPs in a BPP; in other embodiments, BCPs consisting of therapy-resistant cells are intentionally spiked in (added) to the BPP.

In some embodiments, e.g., where the initial heterogeneous populations of cells comprises patient-derived cells, e.g., tumor cells and optionally non-tumor cells (e.g., from a biopsy that includes all or part of a tumor and some surrounding non-cancerous tissue), the methods can include exposing one or more populations of BCPs or BPPs generated from those patient-derived cells to one or more test conditions to determine the effect on the patient cells. Thus, for example, BCPs or BPPs generated from tumor cells can be exposed to test conditions that comprise one or more potential therapeutics (e.g., cancer therapeutic agents), and identity and/or relative abundance of each BCP is determined, e.g., using a method comprising PCR, a hybridization assay, or next-generation sequencing. Thus, an effect on the different kinds of cells in the BPP can be evaluated, e.g., an effect on viability or growth of cells having known genetic, genomic, epigenomic, transcriptomic, proteomic, or other profiles, and/or an effect on viability, proliferation, invasiveness, motility, cell cycle, or other cellular characteristics. For example, the methods can be used to determine responses to medication and potential drug resistance (e.g., to monitor the development or overgrowth of resistant cells, and optionally to identify those populations that later develop resistance). The methods can be used to identify and select therapeutics that provide the most complete response (greatest reduction in affected/tumor cells and/or that overcome resistance (e.g., reduce numbers or don't elicit development of drug-resistant populations of cells) and/or that selectively affect resistant or tumor cells and not normal cells.

These methods can also be used to evaluate drug responses and resistance mechanisms across BCPs and BPPs generated from various cell populations, e.g., primary or non-primary tumor cells, or cells from other disease tissues or models, to screen for new candidate therapeutics or to identify new targets.

### EXAMPLES

The invention is further described in the following examples, which do not limit the scope of the invention described in the claims.

### Example 1. Accounting for Tumor Heterogeneity when Using CRISPR-Cas9 for Cancer Progression and Drug Sensitivity Studies

Gene editing protocols often require the use of a subcloning step to isolate successfully edited cells, the behavior of which is then compared to the aggregate parental population and/or other non-edited subclones. Here we demonstrate that the inherent functional heterogeneity present in many cell lines can render these populations inappropriate controls, resulting in erroneous interpretations of experimental findings. We describe a novel CRISPR/Cas9 protocol that incorporates a single-cell cloning step prior to gene editing, allowing for the generation of appropriately matched, functionally equivalent control and edited cell lines. As a proof of concept, we generated matched control and osteopontin-knockout Her2+ and Estrogen receptor-negative murine mammary carcinoma cell lines and demonstrated that the osteopontin-knockout cell lines exhibit the expected biological phenotypes, including unaffected primary tumor growth kinetics and reduced metastatic outgrowth in female FVB mice. Using these matched cell lines, we discovered that osteopontin-knockout mammary tumors were more sensitive than control tumors to chemotherapy in vivo. Our results demonstrate that heterogeneity must be considered during experimental design when utilizing gene editing protocols and provide a solution to account for it.

Materials and Methods

Cell Lines

McNeu and Met-1 murine mammary carcinoma cells (kind gifts from Drs. Michael Campbell and Johanna Joyce, respectively) were cultured as previously described [30, 31]. Briefly, cells were cultured in DMEM (Gibco) media, supplemented with 10% fetal bovine serum (FBS) and 100 U/ml penicillin-streptomycin at 37° C. under 5% CO₂. Human MDA-MB-435 cells were a generous gift from Dr. Robert Weinberg and were cultured in DMEM:F12 (1:1; Gibco), supplemented with 10% fetal bovine serum and 100 U/ml penicillin-streptomycin at 37° C. under 5% CO₂. All cell lines were validated as Mycoplasma-negative. Human cells were validated using short tandem repeat (STR) profiling (Molecular Diagnostics Laboratory at Dana-Farber Cancer Institute, Boston, Mass.). For mouse cells, the murine strain of origin was confirmed by short tandem repeat analysis (Bioassay Methods Group, NIST).

New Gene Editing Protocol

Clonal subpopulations are generated from parental cell lines by sorting one single cell per well into 96-well plates using a FACSAria II cell sorter (BD Bioscience). Single cell-derived populations are subsequently allowed to proliferate for expansion. A single expanded clone is used for both control and co-transfection with the Cas9/GFP and sgRNA vectors. Select cell populations were seeded into 12-well plates overnight before transfection with 500 ng pCas9_GFP and 500 ng sgRNA expressing plasmids using FugeneHD (Roche). 48 hours after transfection, successfully transfected single cells are isolated by FACS sorting for GFP-positivity using a FACSAria II cell sorter (BD Bioscience) followed by recovery and expansion in 12-well plates for 2-3 days. At confluency, cells were collected for a second round of FACS sorting and single GFP-negative cells were sorted into individual wells in a 96-well plate to ensure that random Cas9/GFP integration did not occur. Following clonal expansion editing was validated using Sanger sequencing and phenotype verification is performed.

To generate luciferase/GFP-positive populations, cells were infected with lentivirus generated from pLV-Luc-IRES-GFP viral plasmids (a generous gift from Dr. Robert Weinberg's lab) and then sorted for GFP-positive populations.

Vector Construction

The human codon-optimized Cas9 expression plasmid pCas9 GFP was a gift from Kiran Musunuru (Addgene plasmid #44719). The sgRNA targeting mouse OPN exon 2 (5′-GTGATTTGCTTTTGCCTATT-3′ (SEQ ID NO:1)) driven by human U6 promoter was synthesized at Eurofin.

Evaluating Target Site Modification by Sanger Sequencing

OPN gene fragments were amplified with the primers OPN-F (5′-GACTTGGTGGTGATCTAGTGG-3′ (SEQ ID NO:2)) and OPN-R (5′-GCCAGAATCAGTCACTTTCAC-3′ (SEQ ID NO:3)) using Phire Animal Tissue Direct PCR Kit (Thermo Scientific). The resulting PCR products were then submitted for Sanger sequencing (Macrogen USA).

Animals and Tumor Studies

Female FVB/NJ mice 7 weeks of age were purchased from Jackson Labs (stock no. 001800). NOD/SCID mice were maintained in-house under aseptic sterile conditions. All experiments were conducted in accordance with regulations of the Children's Hospital Institutional Animal Care and Use Committee (protocol 12-11-2308R), the MIT committee on animal care (protocol 1005-076-08), and Brigham and Women's Hospital animal care protocol committee (2017N000056). Mice were 8-9 weeks of age at the time of study initiation. All efforts were made to minimize animal suffering. Animal facility personnel monitored the animals daily, checking for levels of food, water, and bedding in each cage. Mice were also physically checked three times a week by the investigators. The basic animal maintenance included housing the mice in cages (five per cage) with sufficient diet, water and bedding and cages were cleaned and sanitized on a regular basis. Investigators strictly adhered to approved protocols for humane endpoints; if any animal became severely ill prior to an experimental endpoint, that animal would be euthanized. Humane endpoints were defined as follows: ≥20% weight loss, rough hair coat, jaundice and/or anemia, coughing, labored breathing, nasal discharge, neurological signs (frequent seizure activity, paralysis, ataxia), prolapse, self-induced trauma, any condition interfering with eating or drinking, excessive or prolonged hyperthermia or hypothermia, tumor size ≥1.5 cm³ in volume. Animals were randomly assigned to treatment groups and no animals were excluded from analysis.

For tumor studies, murine mammary carcinoma cells were injected orthotopically, using a total of 10⁵ or 10⁶ McNeu cells, or 2.5×10⁴ or 2.5×10⁵ Met-1 cells implanted into the fourth mammary fat pad of 7-10 week old female FVB mice. Where indicated, either 1×10⁵ or 1×10⁶ cells of the McNeuA parental cell line were implanted subcutaneously. 2.5×10⁵ human MDA-MB-435 cells were injected subcutaneously into 8-10 week old female NOD-SCID mice. Thereafter, tumors were monitored and measured using calipers with volume calculated as 0.5(length×width²).

For the Met-1 metastasis assay, mice received tail vein injections with 10⁶ cells of luciferase-labeled Met-1 cells suspended in 100 μl of sterile phosphate-buffered saline. Pulmonary metastases were monitored weekly by bioluminescent imaging using the Spectrum Imaging System and Living Image software (Caliper Life Sciences, Inc.). Prior to imaging, mice were intraperitoneally administered 150 mg/kg D-luciferin (Perkin-Elmer) and were anesthetized using isoflurane inhalation. Luminescent signal was detected for the regions of interest as radiance (p/sec/cm²/sr) and analyzed using the Living Image Software Version 4.1 (Caliper Life Sciences). Lungs were fixed and stained using Hematoxylin/Eoisin and metastases were classified as multi- or single-focal and were counted manually on 3 separate sections spaced 50 microns apart per mouse. Total lung area was quantified using Cell Profiler and metastases counts were normalized total lung area.

Chemotherapy

For AC-T chemotherapy trials, 2.5×10⁵ Met-1 Luc/GFP cells were injected into the mammary fat pad of 6-8-week-old female FVB mice. Doxorubicin (Teva), paclitaxel (Hospira), and cyclophosphamide (Sigma) were diluted in PBS for in vivo experiments. Mice were treated with two to four doses of 5 mg/kg doxorubicin, 10 mg/kg paclitaxel, and 120 mg/kg cyclophosphamide administered every two weeks. Doxorubicin was administered via retro-orbital injection, and paclitaxel and cyclophosphamide were administered via intraperitoneal injection.

For studies investigating the role of OPN in chemotherapeutic response, 2.5×10⁴ WT or OPN KO tumor cells were injected into the mammary fat pad of 6-8-week-old female FVB mice. When established tumors reached 60-80 mm³ in volume, treatment was initiated. Four treatment arms were included: vehicle control (PBS) on WT or OPN KO cohorts or one dose of paclitaxel (10 mg/kg), doxorubicin (5 mg/kg) and cyclophosphamide (120 mg/kg) by intraperitoneal injection (paclitaxel and cyclophosphamide) and retro-orbital injection (doxorubicin) on WT or OPN KO cohorts. Tumor growth was monitored using caliper measurements. Average tumor mass at sacrifice was measured and is presented as the average±standard error of mean.

Osteopontin ELISAs and Western Blotting

To assess circulating secreted murine osteopontin (mOPN) or human osteopontin (hOPN) protein levels, whole blood was collected in EDTA-coated tubes (VWR) and centrifuged at 1.5×g for 8 minutes to isolate plasma. mOPN and hOPN concentrations were determined by ELISA according to manufacturer's instructions (R&D) and analyzed using a plate reader (Molecular Device).

To quantify secreted mOPN levels in conditioned medium, cells were grown to 80-90% confluence in growth medium containing 10% FBS. Then the medium was replaced with serum-free medium and was collected 24 hours later. mOPN levels in conditioned media were quantified by ELISA or western blotting.

Whole cell lysates were prepared following culture in the presence or absence of brefeldin A (used to prevent the secretion of OPN and ensure detection of protein expression). Cell lysates or concentrated conditioned medium were subjected to SDS-PAGE on 12% gels and then transferred onto a polyvinylidenedifluoride membrane, which was incubated with mouse anti-OPN (final dilution: 1:200, Clone AKm2A1, Santa Cruz Catalog #sc-21742, mouse monoclonal antibody raised against recombinant OPN of mouse origin, references with validation available on manufacturer's datasheet) antibody at 4 C overnight. After being washed, membranes were incubated with horseradish peroxidase-conjugated anti-mouse IgG for 1 hour. The enzyme bound to OPN was visualized using the SuperSignal™ West Pico Chemiluminescent kit (ThermoFisher). The blot was then stripped and incubated with rabbit anti-mouse (3-actin antibody as a loading control (final dilution: 1:1000, Rockland Catalog #600-401-886, rabbit polyclonal antibody raised against human beta-actin, references with validation available on manufacturer's datasheet).

Immunohistochemistry, Immunofluorescence and Microscopy

Formalin-fixed, paraffin embedded tissues were sectioned onto ProbeOn Plus microscope slides (Fisher Scientific) and immunohistochemistry was performed as described [21]. For the OPN immunohistochemistry studies, anti-OPN (final dilution: 1:200, Maine Biotechnology Services Catalog #, MAB197P, mouse monoclonal antibody raised against recombinant OPN of human origin, [44]) was used and was detected using the Vector ABC kit (Vector Laboratories, Burlingame, Calif., USA). For immunofluorescence, anti-OPN (final dilution: 1:50, Clone AKm2A1, Santa Cruz Catalog #sc-21742, mouse monoclonal antibody raised against recombinant OPN of mouse origin, references with validation available on manufacturer's datasheet) was used and was detected using a goat anti-mouse IgG AF549 conjugated secondary antibody (final dilution: 1:1000, Invitrogen Catalog #A11032, polyclonal, references with validation available on manufacturer's datasheet). Nuclei were counterstained with DAPI (Invitrogen). Images were captured with identical exposure and gain using a Nikon Eclipse Ni microscope.

In Vitro Chemosensitivity Studies

4,000 Met-1 cells were plated in quadruplicates in 96-well plates containing growth media. The next day, vehicle (PBS) or chemotherapy (doxorubicin: 0.33 nM-2.2 ∝M; paclitaxel: 14 μM-160 μM) was added to the plate and incubated for 72 hours. ATP levels were quantified as a surrogate measure for viability (CellTiter-Glo, Promega) using a luminometer (Perkin-Elmer).

Statistical Analyses

Data are represented as mean±SEM and analyzed by ANOVA, Student's t-test, and/or Mann-Whitney test as indicated using GraphPad Prism 7.0, unless otherwise stated. P<0.05 was considered statistically significant. Error bars represent standard deviation unless otherwise indicated.

Results

Selection of Her2+ and Estrogen Receptor-Negative Mammary Carcinoma Models

We aimed to design an approach that would enable us to generate appropriately matched control and CRISPR/Cas9 knockout cell lines, while taking into account the inherent functional heterogeneity present in nearly all breast tumors and tumor-derived cancer cell lines. We hypothesized that results from studies employing standard CRISPR/Cas9 approaches, which often require a subcloning and/or selection step, would be confounded by subclonal functional heterogeneity.

As a proof of concept, we chose to study Osteopontin (OPN), a protein that we have studied previously and that is relevant for breast cancer metastasis [18-25]. OPN plays an important role in metastasis and survival in many pre-clinical cancer studies, and is positively associated with metastasis as well as reduced progression-free and overall survival in breast cancer patients [24-26]. Additionally, OPN has been shown to play a role in chemoresistance in some cancer types types [18, 20, 22, 27-29], but it is unclear whether this is also true of breast cancer.

Hence, we determined that the breast cancer models that we would employ must meet the following criteria: secretion of detectable levels OPN both in vitro and in vivo, capacity to form primary and metastatic tumors in vivo, evidence of heterogeneity, and responsiveness to chemotherapy.

Transgenic mice that specifically overexpress oncogenic proteins in the mammary fat pad are commonly employed both for the study of spontaneous breast tumors and as a source for murine breast cancer cell lines that can be allografted orthotopically in immunocompetent animals. In this study, we utilized two such murine breast cancer cell lines: McNeuA, a HER2⁺ breast cancer cell line derived from a spontaneously arising mammary carcinoma in a MMTV-neu transgenic mouse [30], and Met-1, an estrogen receptor-negative (ER⁻) breast cancer cell line derived from a mammary carcinoma in a MMTV-PyMT transgenic mouse (FVB/N-Tg(MMTV-PyVmT) [31].

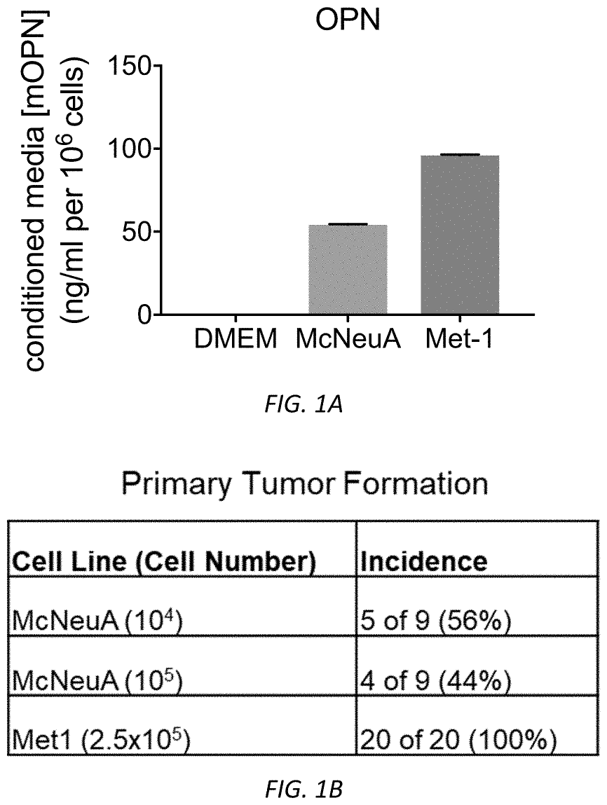

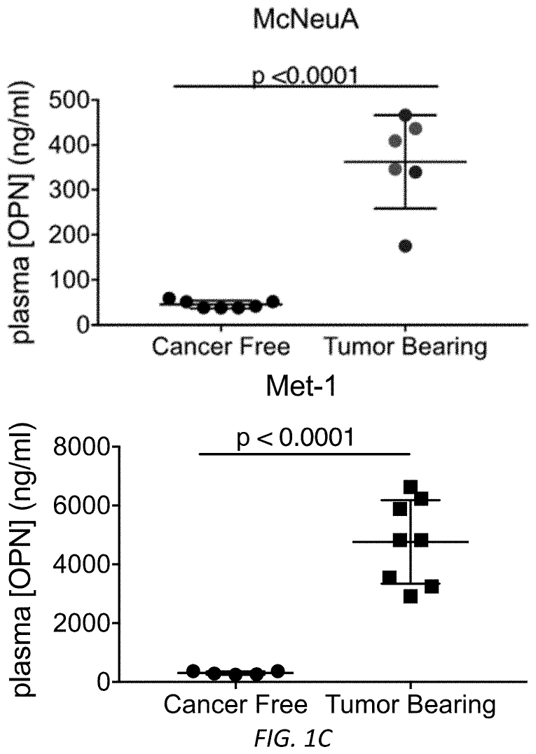

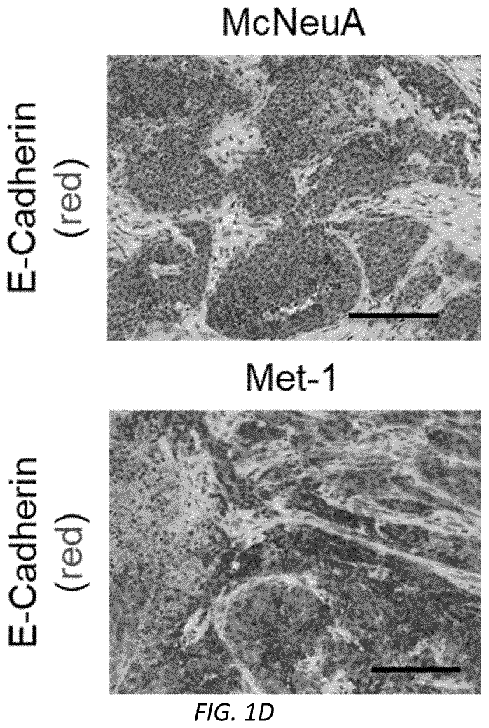

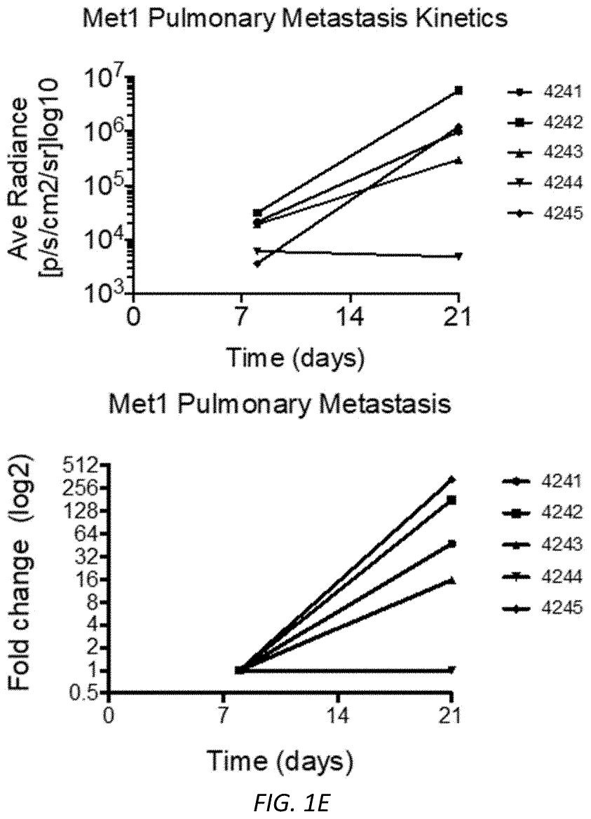

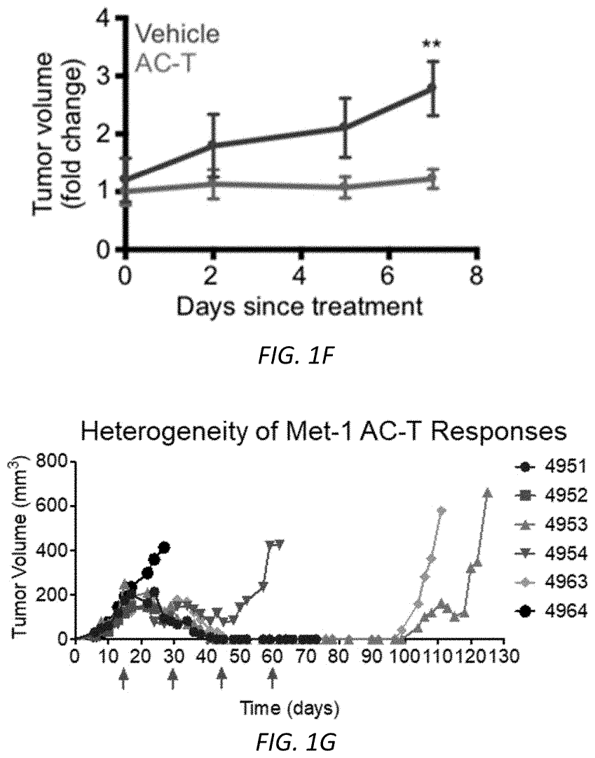

Characterization of McNeuA and Met-1 cell lines demonstrated their potential as models for this study, as they secreted detectable levels of OPN in culture as measured by ELISA (FIG. 1A). Both cell lines efficiently formed primary tumors following injection into FVB mice (FIG. 1B). While both cell lines formed tumors that had an average mass of 2.3 g at the experimental end points (30 days for Met-1 and 90 days for McNeuA, or when tumors reached 1.5 mm³), the McNeuA tumors exhibited more variability in both their tumor incidence and final tumor mass.

In both models, the tumor bearing mice had significantly elevated plasma levels of OPN relative to cancer-free cohorts whereby average OPN levels were 8-fold and 15-fold higher in the McNeuA and Met1 tumor-bearing mice, respectively, at end stage (FIG. 1C). Interestingly, plasma OPN levels positively correlated with the final tumor mass in mice bearing the McNeuA tumors. Immunohistochemical analysis of the recovered tumors revealed intratumoral heterogeneity for the epithelial marker, E-cadherin (FIG. 1D).

Previous studies have demonstrated that both of these cell lines are capable of forming lung metastases [30, 31]. We were particularly interested in the Met-1 cell line, as women with metastatic ER-breast cancer most often experience pulmonary metastases [32]. We confirmed that the Met-1 cells formed pulmonary metastases, with 4 of 5 mice experiencing increased metastatic burden (˜15-300-fold increases) over the experimental time course (FIG. 1E).

We next tested responsiveness of Met-1 mammary carcinoma to combination doxorubicin (A), cyclophosphamide (C), and paclitaxel (T) chemotherapy, AC-T, a standard of care chemotherapy regimen for breast cancer patients with ER-negative disease. We first tested the sensitivity of Met-1 cells to doxorubicin and paclitaxel in vitro and performed an initial in vivo experiment to identify a therapeutically relevant, well-tolerated combinatorial dose.

Treatment with both doxorubicin and paclitaxel significantly decreased viability of Met-1 cells in vitro. Cyclophosphamide, a pro-drug, requires activation into cytotoxic metabolites by liver enzymes in vivo and was therefore not tested in vitro. In vivo, a neoadjuvant combination dose of doxorubicin (5 mg/kg), paclitaxel (10 mg/kg), and cyclophosphamide (120 mg/kg) was well tolerated (no weight loss; data not shown) and had a cytostatic effect on Met-1 tumor growth (FIG. 1F).

To more closely emulate the clinical dosing regimen of AC-T chemotherapy, mice with Met1 mammary carcinoma were administered neoadjuvant AC-T every 2 weeks for 4 cycles. Interestingly, individual mice bearing Met-1 tumors exhibited differential responses to treatment, and in some cases, mice that initially experienced complete tumor regression eventually experienced local recurrence (FIG. 1G).

Collectively, our analyses indicated that the McNeuA and Met-1 cell lines met our criteria of OPN secretion in vitro and in vivo, efficient formation of primary orthotopic tumors, and evidence of phenotypic and functional heterogeneity in vivo. Moreover, the Met-1 cells met the criteria of metastatic capacity and, chemosensitivity. Hence, the McNeuA and Met-1 cell lines were ideal for our investigation into the effect of tumor heterogeneity on the generation of appropriately matched control and OPN-KO cell lines.

Heterogeneity Between Subclonal Populations Derived from McNeuA and Met-1

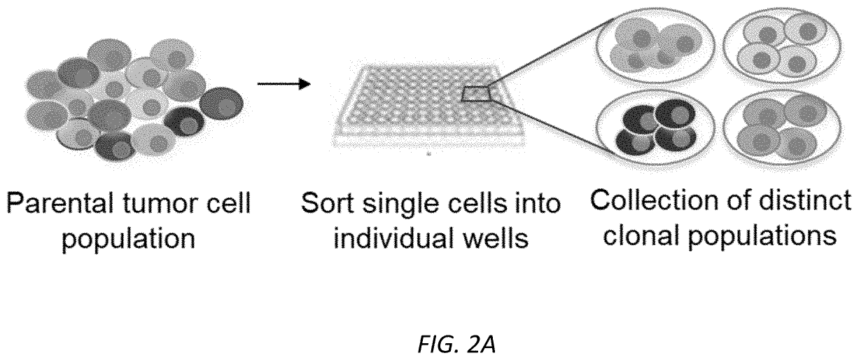

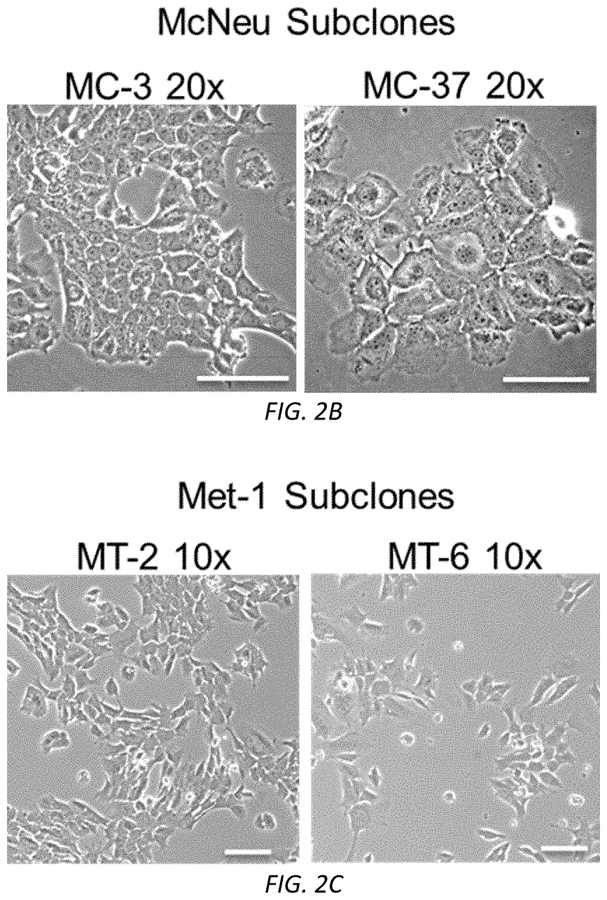

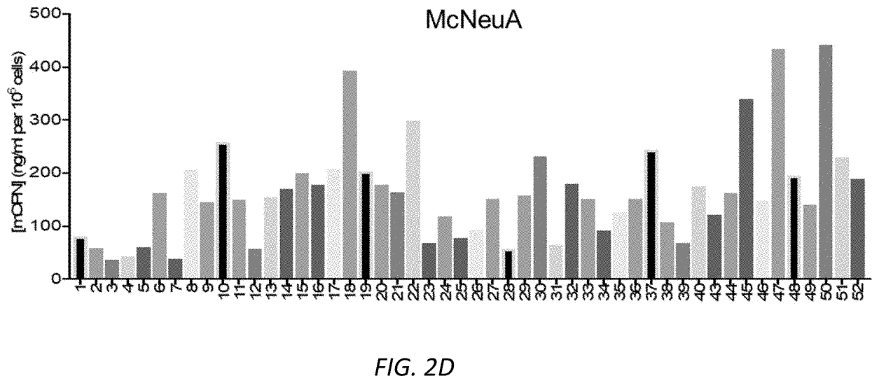

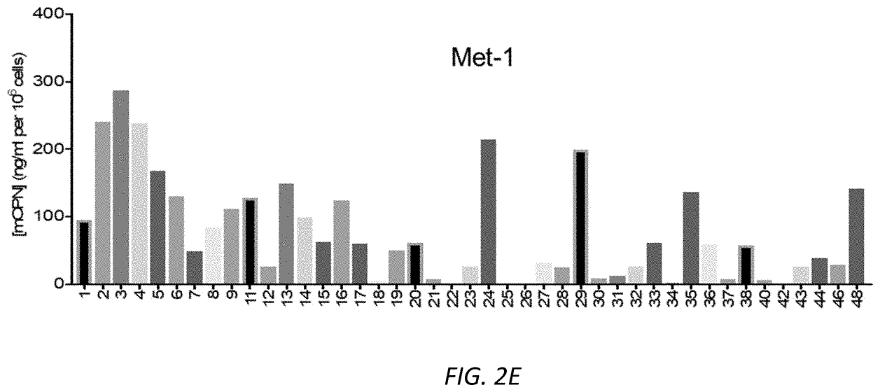

In order to better understand whether the inherent phenotypic heterogeneity of the McNeuA and Met-1 cells lines would potentially confound the results of an OPN-knockout study, we generated single cell-derived subclonal populations from both the McNeuA (50 clones) and Met-1 (42 clones) parental cell lines (FIG. 2A). The various subclonal populations exhibited morphological heterogeneity, displaying a range of epithelial and mesenchymal phenotypes in culture (FIG. 2B,2C). Cell size also appeared to vary between subclones for each given cell line (FIG. 2B,2C).

Levels of OPN secreted in vitro by the McNeuA and Met-1 subclones varied considerably. The McNeuA subclones secreted a range of OPN from 37.5-442.1 ng/ml per 10⁶ cells (FIG. 2D), while the Met1 subclones exhibited a range from no detectable OPN to 287.6 ng/ml per 10⁶ cells (FIG. 2E). Importantly, a number of individual subclones secreted levels of OPN that differed significantly from their respective parental population. For example, OPN was 6-8-fold higher in some McNeuA subclones (MC-18, MC-22, MC-45, MC-47, MC-50) and 2.5-3-fold higher in some Met1 subclones (MT-2, MT-3, MT-4) than the parental populations (FIGS. 1A and 2D,2E). Likewise, OPN was undetectable in some of the Met1 cells (MT-18, MT-22, MT-25, MT-26, MT-40, MT-42) (FIG. 2E). We observed similar heterogeneity of OPN secretion from clonal populations that we derived from a human melanoma cell line, MDA-MB-435, suggesting that this phenomenon is not limited to murine cell lines or cancer type.

Taken together, these results highlighted the phenotypic heterogeneity that exists within tumor-derived breast carcinoma populations in vitro. We therefore wondered if different clones would perform differently in vivo as well.

McNeuA and Met-1 Derived Clonal Populations Behave Differently In Vivo

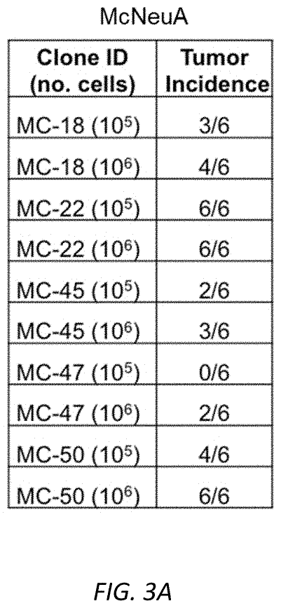

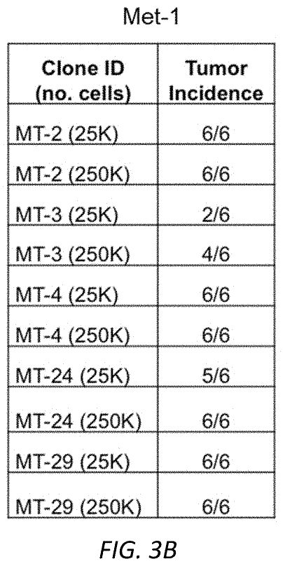

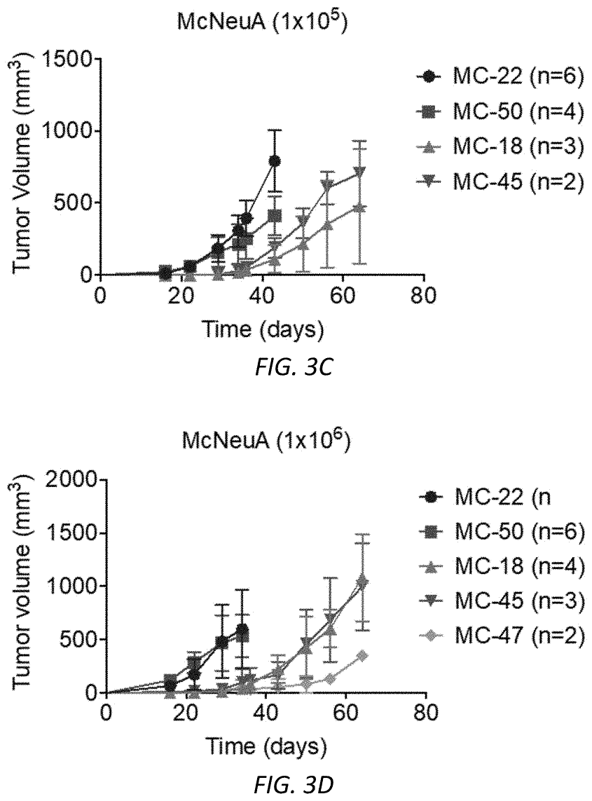

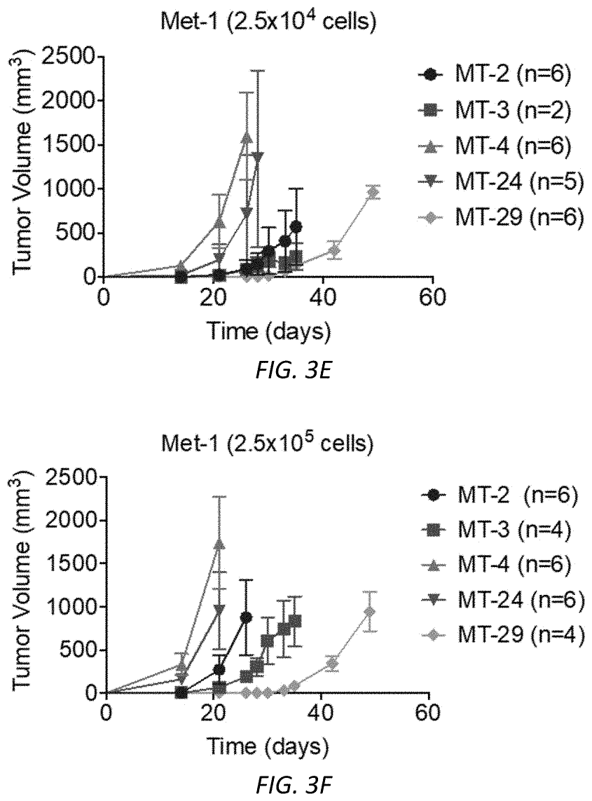

To understand whether various subclones that displayed different phenotypes in vitro would also display functional heterogeneity with respect to tumorigenesis, we injected cohorts of FVB mice orthotopically with various McNeuA or Met-1 subclonal populations and monitored tumor growth parameters over a course of 64 or 49 days, respectively. We chose to use five subclones from each cell line that secreted the highest levels of OPN (MC-18, MC-22, MC-45, MC-47, MC-50 and MT-2, MT-3, MT-4, MT24, MT-29) (FIG. 2D,2E). We injected either 10⁵ or 10⁶ cells of each McNeuA subclone and 2.5×10⁴ or 2.5×10⁵ cells of each Met-1 subclone. Among the McNeuA subclones, a subset of clones (MC-22 and MC-50) formed tumors with 100% incidence, while another (MC-47) failed to form tumors in any mice, and incidence was only slightly higher when more cells were injected (FIG. 3A). Similarly, Met-1 subclones also exhibited variable tumor incidence with 4 of 5 subclones (MT-2, MT-4, MT-24, and MT-29) forming tumors with ˜100% incidence while one subclone (MT-3) had reduced incidence to 50-66%, depending on the numbers of cells injected (FIG. 3B).

Those clones that formed tumors displayed variability in latency and growth kinetics. For example, latency and growth kinetics were not statistically different between MC-22 and MC-50 when 10⁶ cells were injected (FIG. 3D); however, growth kinetics differed significantly between these clones at 10⁵ (p<0.0001, FIG. 3C). The subclonal populations also exhibited differences in latency. For example, when 10⁶ cells were injected, MC-22 and MC-50 had latencies of ˜20 days, MC-18 and MC-45 had latencies of ˜40 days, and MC-47 had a latency of ˜60 days (FIG. 3D).

Similarly, the growth kinetics of the Met-1 subclonal populations was also variable. When 2.5×10⁴ cells were injected, at the 28 day time point (when the MT-4 cohort had reached its endpoint), its growth kinetics were significantly different from the MT-2, MT-4, MT-24 and MT-29 subclones (p<0.0002, FIG. 3E). The Met-1 subclones also had different latencies, with the MT-4 and MT-24 clones having shorter latencies than the other subclonal populations when either 2.5×10⁴ or 2.5×10⁵ cells were injected (FIG. 3E,3F).

The subclones derived from the human melanoma cell line also varied in incidence of subcutaneous tumor formation in NOD-SCID mice, with some clones (i.e. 11, 28, 29, 30) unable to form tumors in vivo. Moreover, tumor mass at the experimental end point varied considerably among these subclones.

Critically, a number of individual subclonal populations from each tumor model exhibited different tumor formation capabilities than the respective bulk parental population from which they were derived. For example, while the parental Met-1 tumor cell line formed orthotopic tumors with 100% incidence, the MT-3 subclonal cell line formed tumors with only 60% incidence when the same number of cells was injected (FIGS. 1B and 3B). This was also true of the human xenograft model.

These observations revealed the considerable subclonal heterogeneity that exists within human and murine mammary carcinoma cell lines and that the behavior of individual subclones differs from their respective parental populations.

Evidence that Identification of Proper Controls is Necessary for Correct Interpretation of Experimental Findings

Traditional CRISPR/Cas9 editing protocols begin with infection or transfection of the bulk parental population [3-7]. For this reason, the unedited or mock-infected parental cell line is typically used as a control. Due to the inefficiency of infection and/or editing in certain cell lines (especially tumor cell lines that are hyperploid), there is often a subclonal selection step that follows the initial infection and then a validated, edited subclone is used for subsequent experimentation. Our initial characterizations of the McNeuA and Met-1 parental and subclonal populations demonstrate why one must use caution when considering this commonly used approach.

In some scenarios, subclonal heterogeneity could confound interpretation of knockout efficiency. For example, 23% of the Met-1 subclones have low or no detectable secreted OPN (FIG. 2E). Hence, if one randomly selected one of these clones (e.g. MT-42) and evaluated the functional success of the OPN KO by comparing its OPN secretion levels to that of the parental Met-1 cell line, a failed knockout attempt or false positive result could be overlooked.

In another scenario, if the clonal population that was selected after CRISPR/Cas9 OPN-knockout happened to be clone MT-3 and its orthotopic tumor penetrance was compared to that of the parental Met-1 population, then one could erroneously interpret the necessity of OPN for primary tumor formation, when in fact this clone, prior to OPN knockout, already inherently forms tumors with lower incidence (˜66%) than the parental population (100%) (FIGS. 1B and 3B).

Likewise, comparing two subclonal populations, even those that secrete similar levels of OPN and form tumors with the same incidence, could also lead to spurious results. For example, if one randomly selected MT-29 as an OPN KO clone and MT-4 as a control, then incorrect conclusions could be drawn about the role of OPN in tumor growth. This is because prior to OPN KO, both clones express similar levels of OPN (˜225 ng/ml; FIG. 2E) and form tumors with similar incidence (FIG. 3B) but MT-29 inherently exhibits significantly longer latency and reduced growth kinetics than MT-4 (FIG. 3E,3F). The same holds true for MC-18 and MC-50, which secrete similar levels of OPN (˜400 ng/ml; FIG. 2D), but incidence of tumor formation after injecting 10⁶ cells is ˜17% for MC-18 and 100% for MC-50 (FIG. 3A). Hence, the chances of randomly selecting functionally equivalent clones—such as MC-22 and MC-50, which secrete similar levels of OPN (>250 ng/ml; FIG. 2D), form tumors with similar incidence (100%; FIG. 3A), and display similar growth kinetics (FIG. 3B)—are low without extensive characterization of individual clones prior to gene editing.

Our results provided evidence that neither the parental population nor other subclones would represent an appropriately matched wild-type control for a CRISPR/Cas9 knockout cell line that was selected after the gene editing step. The only appropriate control would be to compare the behavior of edited and unedited cells derived from the same clonal population. We therefore concluded that a modified strategy should be developed to account for heterogeneity and enable the generation of appropriately matched cell lines.

Generating spp1 Knockout Clonal Populations Via CRISPR/Cas9

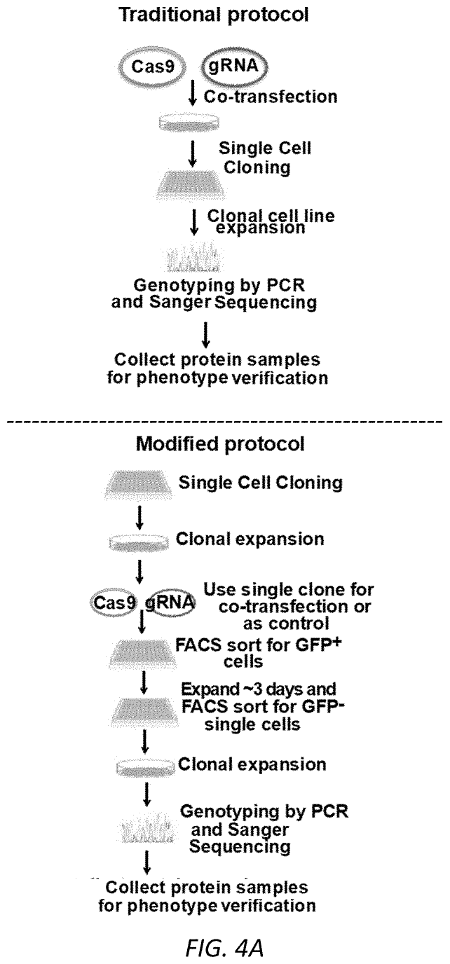

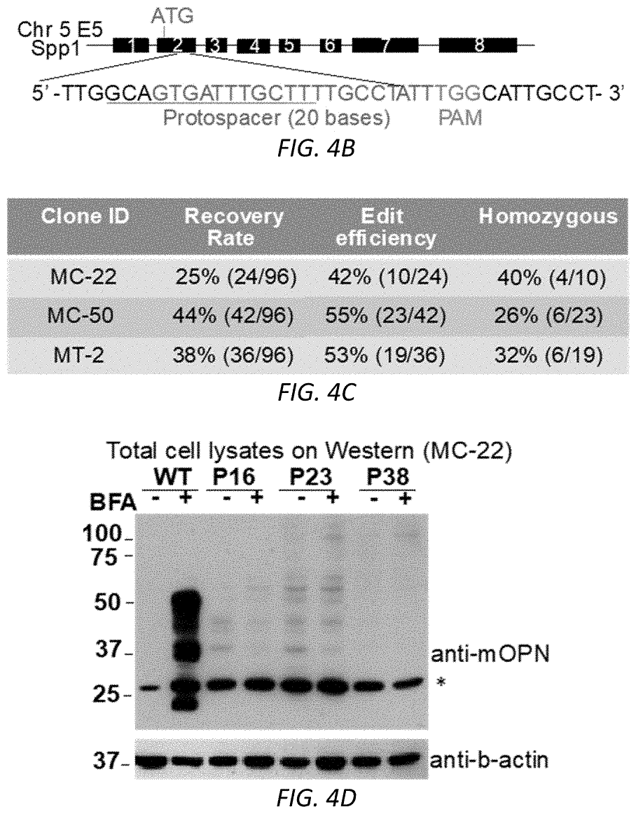

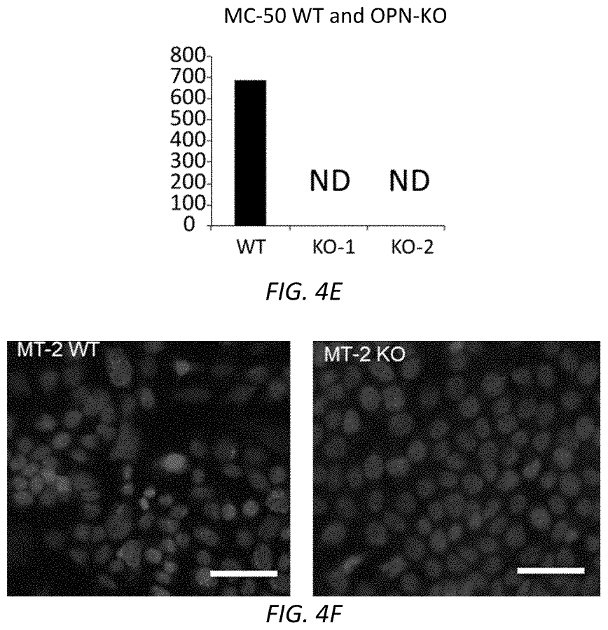

One would not have known a priori about differences in subclonal biological phenotypes and experimental outcomes by taking traditional approaches to gene editing. Therefore, we developed a modified CRISPR-Cas9 editing protocol for generating matched control and knockout cells. Appropriate subclonal populations that we had generated and characterized were chosen for CRISPR/Cas9 gene targeting based on desired biological properties of high intrinsic levels of OPN secretion and orthotopic tumor incidence of 100%. We identified three clonal populations that fit these criteria: MC-22, MC-50, and MT-2 (FIGS. 2D,2E and 3A,3B). Hence, in contrast to traditional CRISPR/Cas9 protocols, we used single cell-derived subclonal populations that we had generated prior to CRISPR/Cas9 gene targeting (FIG. 4A).

We used our modified CRISPR/Cas9 editing strategy to delete the spp1 gene (which encodes Osteopontin) in each subclonal population in order to generate OPN KO cell lines. To do so, the individual subclonal populations were transiently co-transfected with a human codon-optimized spCas9-2A-GFP fusion protein expression plasmid (Addgene plasmid #44719) and a plasmid harboring a sgRNA targeting exon 2 of spp1 (FIG. 4B). After 24 hours, the GFP-positive (and therefore successfully transfected) Cas9-expressing cells from each subclonal population were collected by FACS and allowed to expand in culture for at least six doublings (˜3 days) (FIG. 4A). By giving transfected cells more time to recover from FACS sorting, we observed improved single cell cloning recovery rates for the MC-22, MC-50, and MT-2 subclones (respectively 42%, 55%, and 53%, FIG. 4C) compared to transfected cells that were directly sorted as single cells, in which the recovery rate was ˜5% in an initial trial (data not shown). The higher colony recovery rate and enrichment of Cas9 expressing cells during the first sorting step allowed us to achieve both higher editing efficiency and more homozygously edited clones (FIG. 4C).

Due to the transient nature of our transfection protocol, only cells in which the Cas9-GFP fusion protein had been randomly integrated would maintain GFP expression past this point. In order to avoid random integration of the Cas9 expression plasmid into the genome, a second round of single-cell sorting by FACS was employed to isolate cells that had not undergone a Cas9 integration event by sorting and selecting for GFP-negative cells (FIG. 4A). Single cell-derived subclones were then expanded in culture.

We next employed Sanger sequencing to identify the edited subpopulations from among the recovered subclones. Of the recovered subclones from the MC-22, MC-50, and MT-2 lines, a subset of the single cell clones contained either a hemizygous or homozygous mutation in the spp1 gene, representing editing efficiencies of 42%, 55%, and 53%, respectively (FIG. 4C). We found that MC-50 clone is hyperploid for the chromosome region containing spp1 based on partially edited clones' sequencing result and this observation was further validated by genotyping these clones using TA cloning and Sanger sequencing (data not shown). Between 26-40% of the successfully edited clones contained homozygous mutations (FIG. 4C).

We validated loss of OPN protein expression in each of the OPN KO clones compared to its appropriately matched control using western blotting, ELISA of conditioned media, or immunocytochemistry. We observed no detectable OPN protein (FIG. 4D-4F), demonstrating that our CRISPR/Cas9 editing strategy was successful and we had generated authentic OPN KO subclonal cell lines.

Osteopontin is Dispensable for Primary Tumor Growth

Most studies, including our own, report that OPN is dispensable for primary tumor growth, but is critical for metastasis due to its effects on tumor cells, the host systemic environment, and the tumor microenvironment [19, 21, 23]. Therefore, successful generation of appropriately matched KO and WT cell lines should also reflect these properties (e.g., loss of OPN should have no effect on primary tumor growth, but should alter metastatic ability). This makes OPN an ideal protein to test our concept because its dispensability for primary tumor growth means that WT and OPN KO clones should exhibit similar primary tumor growth kinetics and incidence. Therefore, we tested the tumor formation capabilities of the matched clones.

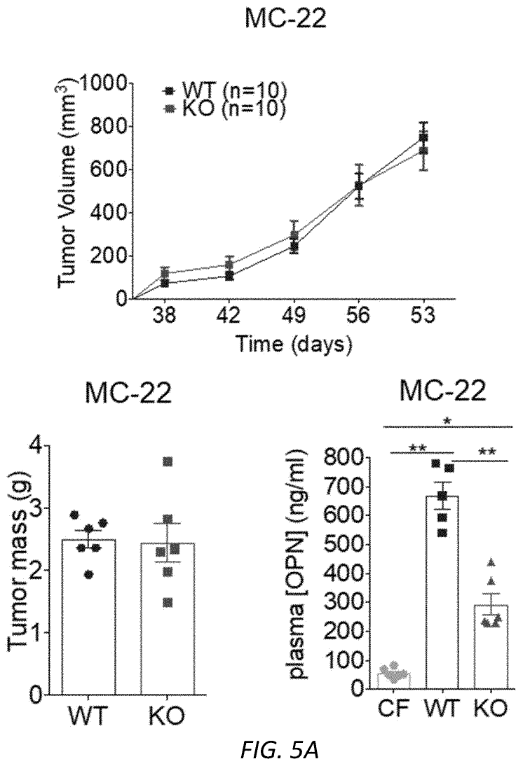

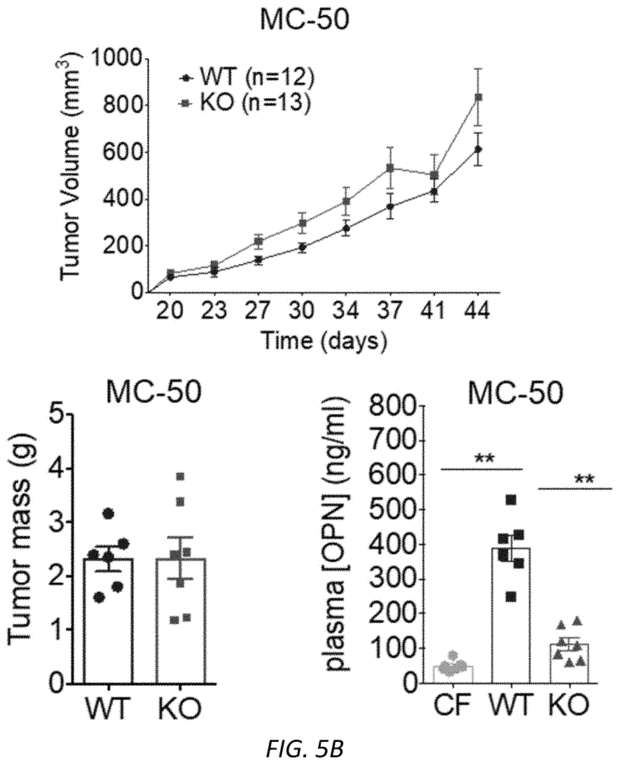

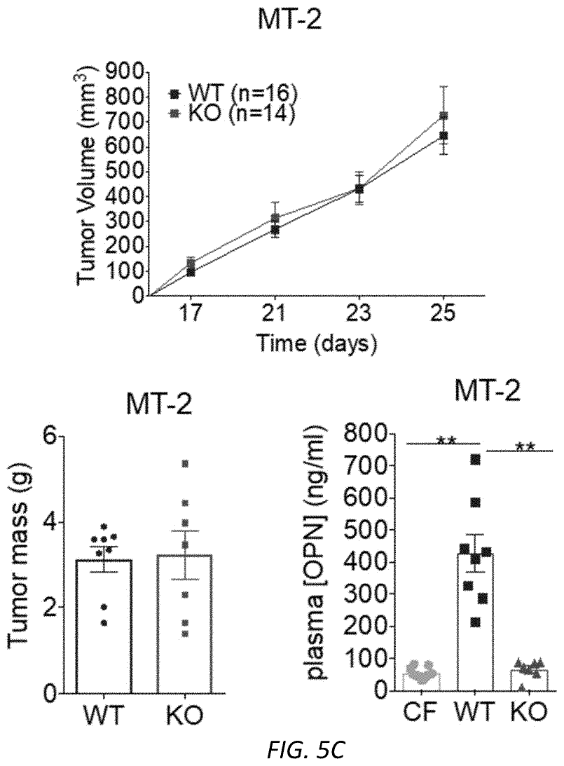

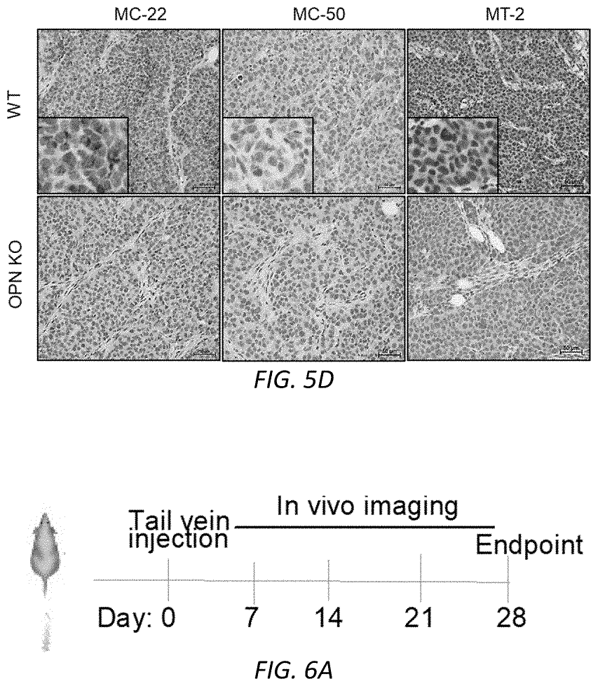

WT and OPN KO MC-50 cells (2×10⁵), MC-22 cells (1×10⁵) or MT-2 cells (2.5×10⁴) were orthotopically injected into FVB mice and were allowed to grow until tumors reached ˜1 cm³. Loss of tumor-derived OPN did not significantly affect growth kinetics or the final mass of any of the tumors derived from matched subclonal cell lines (FIG. 5A-5C). In fact, there were no significant differences in any other tumor growth parameters (FIG. 5A-5C) or spleen mass between cohorts bearing WT and the respective matched OPN KO tumors.

As a control, we tested the concentration of circulating plasma mOPN in the tumor-bearing mice and cancer-free controls. As expected, mOPN plasma levels were elevated in the mice bearing WT tumors relative to the cancer-free cohort, and plasma OPN levels were significantly reduced in the mice bearing KO tumors relative to WT (FIG. 5A-C). Plasma OPN levels from the cohorts of mice bearing MC-50 and MT-2 OPN KO tumors were not significantly different from their respective cancer-free cohorts (FIG. 5A-5C). However, plasma OPN from mice bearing MC-22 KO tumors was significantly higher than the cancer free controls (FIG. 5A), suggesting that clone MC-22 may in fact induce an elevation in host-derived OPN.

It is important to note that if we had used the parental McNeuA cell line as a WT control rather than the appropriately matched WT MC-22 cell line, we would have failed to see a significant difference in the circulating OPN levels between cohorts (FIG. 4B). This observation would not have been possible using a traditional CRISPR/Cas9 gene editing protocol, highlighting once again the strength of our system and the necessity of using appropriately matched control cell lines in knockout studies.

Finally, we visualized OPN expression in the tumors that formed in each cohort using immunohistochemical staining. We observed positive staining for OPN in the WT MC-22, MC-50, and MT-2 tumors, but did not detect any OPN⁺ cells in the corresponding OPN KO tumors (FIG. 5D), confirming that the OPN KO was successful. These observations provided further evidence that any circulating OPN detected in mice injected with the OPN-KO clones (FIG. 5A-5C) was host-derived rather than tumor derived.

Together, these results demonstrated that our modified CRISPR/Cas9 gene editing protocol can be successfully used for studies examining the role of a gene in primary tumor outgrowth.

Loss of Osteopontin Reduces Multifocal Metastatic Outgrowth

Osteopontin is considered a biomarker for tumor progression and is detected at higher levels in more aggressive tumors than their low-grade counterparts, is elevated in the serum of patients with metastatic disease, and is included in gene lists predicting poor prognosis for many cancer types [25, 33-39]. Although OPN is most often dispensable for primary tumor growth, OPN is necessary for metastasis [21, 40-42].

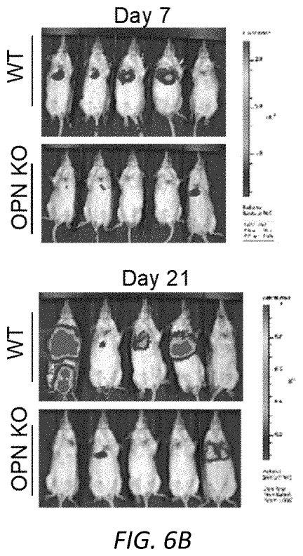

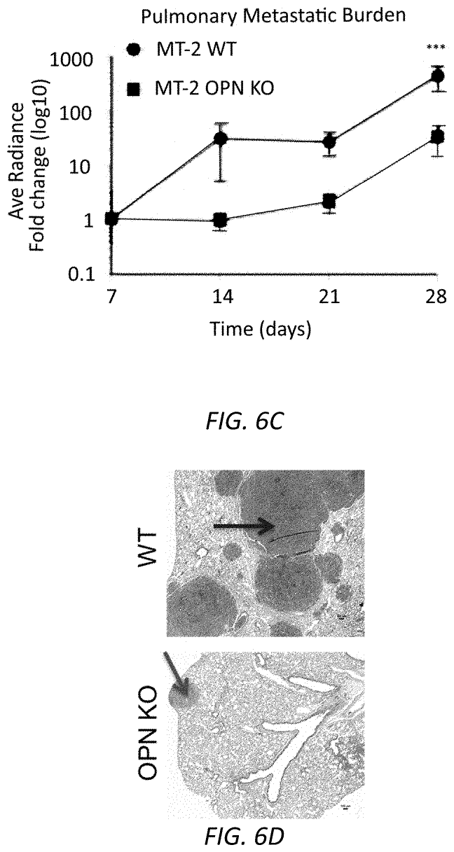

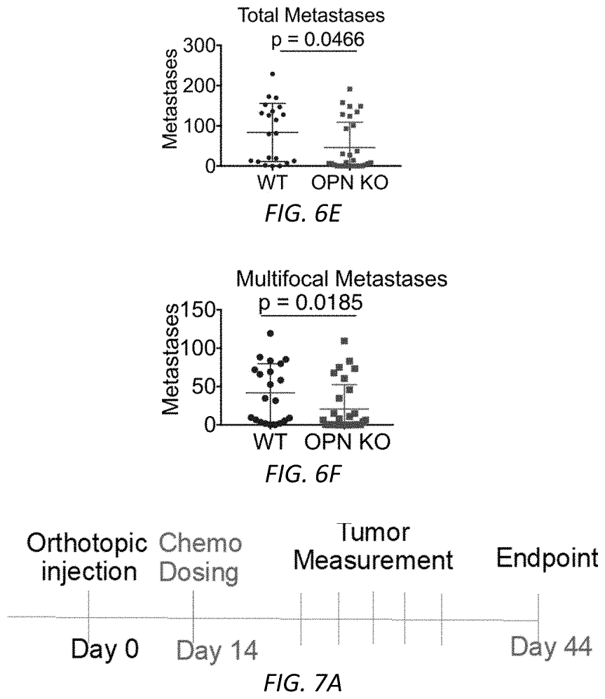

Met-1 cells are highly metastatic [31] (FIG. 1E) and therefore serve as an ideal pre-clinical model of ER-negative disease to test whether our CRISPR/Cas9 system is useful for metastasis studies. To address this question, we labeled the MT-2 WT and MT-2 OPN KO cell lines with a dual GFP/luciferase reporter and injected the labeled cells intravenously via the tail vein into cohorts of mice (FIG. 6A). Metastasis formation was monitored using bioluminescent in vivo imaging at weekly intervals.

Metastatic burden was decreased in the MT-2 OPN KO cohort relative to that of the MT-2 WT cohort, as indicated by reduced bioluminescent signal over the course of the experiment (p=0.0085 at day 7, p>0.05 for all other time points; FIG. 6B,6C). As further confirmation, we analyzed H&E lung sections at the experimental end point and quantified the numbers of single and multifocal metastases. There were significantly fewer total and multifocal pulmonary metastases in mice that had been injected with the OPN KO cells compared to mice that had been injected with OPN WT cells (FIG. 6D-6F). Additionally, the average number of single-focus metastatic outgrowths was also reduced in mice in the OPN KO cohort compared to the WT cohort.

Collectively, our results established that by using appropriately matched cells, we could confidently conclude that OPN is necessary for metastatic colonization and that our CRISPR/Cas9 protocol is useful for pre-clinical metastasis studies.

Loss of Osteopontin Enhances Chemosensitivity

Resistance to standard chemotherapies remains a significant clinical problem, particularly for triple-negative breast cancer [43]. In order to interrogate whether OPN contributes to chemoresistance in breast cancer models, we tested the MT-2 WT and KO cell lines for sensitivity to AC-T chemotherapy in vivo.

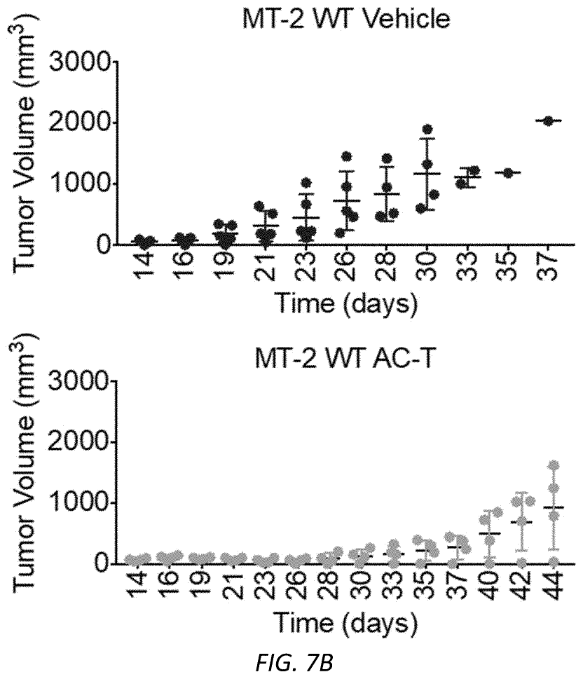

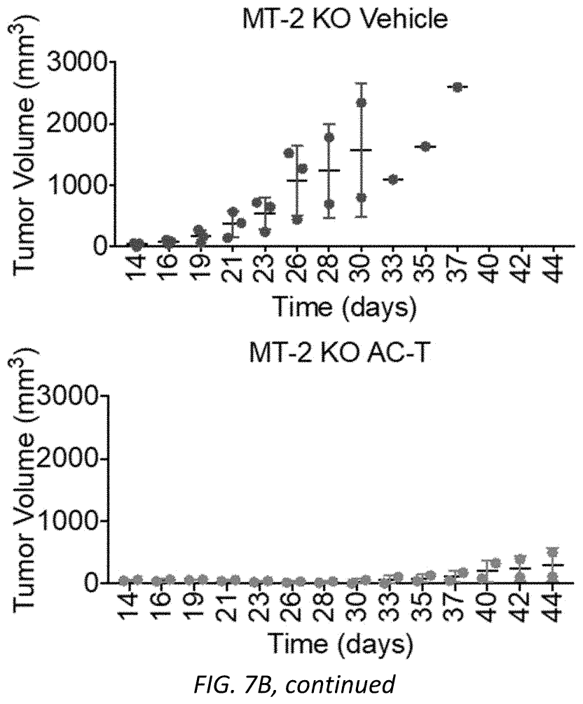

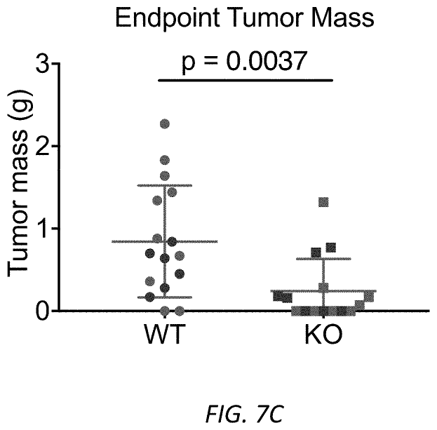

We injected 2.5×10⁴ MT-2 WT or matched OPN KO tumor cells into the mammary fat pads of FVB mice. When established tumors reached ˜60-80 mm³ in volume (14 days), animals were randomized based on tumor volume and enrolled into either vehicle control (PBS) or AC-T chemotherapy treatment cohorts (FIG. 7A).

MT-2 WT and MT-2 KO tumors exhibited sensitivity to AC-T treatment relative to their respective vehicle-treated cohorts (FIG. 7B). However, in response to AC-T, the MT-2 KO tumors exhibited reduced growth kinetics compared to their MT-2 WT counterparts in three independent trials (FIG. 7B). Likewise, final tumor mass was significantly lower in the MT-2 KO treatment cohorts compared to the MT-2 WT treatment cohorts (FIG. 7C). Sensitivity to doxorubicin and paclitaxel was not apparent in vitro. Hence, the enhanced sensitivity observed in vivo could be due to the effects of OPN only on cyclophosphamide resistance, the host microenvironment, or both.

Together, these data established that elimination of OPN expression enhances chemosensitivity of the MT-2 breast cancer population.

### REFERENCES

1. Guernet, A. and L. Grumolato, CRISPR/Cas9 editing of the genome for cancer modeling. Methods, 2017. 121-122: p. 130-137.
2. Doudna, J. A. and E. Charpentier, Genome editing. The new frontier of genome engineering with CRISPR-Cas9. Science, 2014. 346(6213): p. 1258096.
3. Yang, L., et al., CRISPR/Cas9-Directed Genome Editing of Cultured Cells. Curr Protoc Mol Biol, 2014. 107: p. 3111-17.
4. Harrod, A., et al., Genomic modelling of the ESR1 Y537S mutation for evaluating function and new therapeutic approaches for metastatic breast cancer. Oncogene, 2017. 36(16): p. 2286-2296.
5. Neggers, J. E., et al., Identifying drug-target selectivity of small-molecule CRM1/XPO1 inhibitors by CRISPR/Cas9 genome editing. Chem Biol, 2015. 22(1): p. 107-16.
6. Mali, P., et al., RNA-guided human genome engineering via Cas9. Science, 2013. 339(6121): p. 823-6.
7. Ran, F. A., et al., Genome engineering using the CRISPR-Cas9 system. Nat Protoc, 2013. 8(11): p. 2281-2308.
8. McGranahan, N. and C. Swanton, Clonal Heterogeneity and Tumor Evolution: Past, Present, and the Future. Cell, 2017. 168(4): p. 613-628.
9. Swanton, C., Intratumor heterogeneity: evolution through space and time. Cancer Res, 2012. 72(19): p. 4875-82.
10. Burrell, R. A., et al., The causes and consequences of genetic heterogeneity in cancer evolution. Nature, 2013. 501(7467): p. 338-45.
11. Laughney, A. M., et al., Dynamics of Tumor Heterogeneity Derived from Clonal Karyotypic Evolution. Cell Rep, 2015. 12(5): p. 809-20.
12. Gupta, P. B., et al., Stochastic state transitions give rise to phenotypic equilibrium in populations of cancer cells. Cell, 2011. 146(4): p. 633-44.
13. Kreso, A., et al., Variable clonal repopulation dynamics influence chemotherapy response in colorectal cancer. Science, 2013. 339(6119): p. 543-8.
14. Goldberg, D. E., R. F. Siliciano, and W. R. Jacobs, Jr., Outwitting evolution: fighting drug-resistant TB, malaria, and HIV. Cell, 2012. 148(6): p. 1271-83.
15. Sanchez-Romero, M. A. and J. Casadesus, Contribution of phenotypic heterogeneity to adaptive antibiotic resistance. Proc Natl Acad Sci USA, 2014. 111(1): p. 355-60.
16. Chait, R., et al., Pervasive selection for and against antibiotic resistance in inhomogeneous multistress environments. Nat Commun, 2016. 7: p. 10333.
17. Levin-Reisman, I., et al., Antibiotic tolerance facilitates the evolution of resistance. Science, 2017. 355(6327): p. 826-830.
18. Gu, T., et al., Osteopontin is involved in the development of acquired chemo-resistance of cisplatin in small cell lung cancer. Lung Cancer, 2009. 66(2): p. 176-83.
19. Jia, R., et al., Osteopontin facilitates tumor metastasis by regulating epithelial-mesenchymal plasticity. Cell Death Dis, 2016. 7(12): p. e2564.
20. Liu, G., et al., Osteopontin induces autophagy to promote chemo-resistance in human hepatocellular carcinoma cells. Cancer Lett, 2016. 383(2): p. 171-182.
21. McAllister, S. S., et al., Systemic endocrine instigation of indolent tumor growth requires osteopontin. Cell, 2008. 133(6): p. 994-1005.
22. Pang, H., et al., Knockdown of osteopontin chemosensitizes MDA-MB-231 cells to cyclophosphamide by enhancing apoptosis through activating p38 MAPK pathway. Cancer Biother Radiopharm, 2011. 26(2): p. 165-73.
23. Sangaletti, S., et al., Osteopontin shapes immunosuppression in the metastatic niche. Cancer Res, 2014. 74(17): p. 4706-19.
24. Singhal, H., et al., Elevated plasma osteopontin in metastatic breast cancer associated with increased tumor burden and decreased survival. Clin Cancer Res, 1997. 3(4): p. 605-11.
25. Tuck, A. B., A. F. Chambers, and A. L. Allan, Osteopontin overexpression in breast cancer: knowledge gained and possible implications for clinical management. J Cell Biochem, 2007. 102(4): p. 859-68.
26. Sun, Y., et al., Roles of osteopontin and matrix metalloproteinase-7 in occurrence, progression, and prognosis of nonsmall cell lung cancer. J Res Med Sci, 2015. 20(12): p. 1138-46.
27. Luo, S. D., et al., Osteopontin Involves Cisplatin Resistance and Poor Prognosis in Oral Squamous Cell Carcinoma. Biomed Res Int, 2015. 2015: p. 508587.
28. Ding, K., et al., Overexpression of osteopontin promotes resistance to cisplatin treatment in HCC. Oncol Rep, 2015. 34(6): p. 3297-303.
29. Ng, L., et al., Osteopontin Overexpression Induced Tumor Progression and Chemoresistance to Oxaliplatin through Induction of Stem-Like Properties in Human Colorectal Cancer. Stem Cells Int, 2015. 2015: p. 247892.
30. Campbell, M. J., et al., Epithelial and fibroblast cell lines derived from a spontaneous mammary carcinoma in a MMTV/neu transgenic mouse. In Vitro Cell Dev Biol Anim, 2002. 38(6): p. 326-33.
31. Borowsky, A. D., et al., Syngeneic mouse mammary carcinoma cell lines: two closely related cell lines with divergent metastatic behavior. Clin Exp Metastasis, 2005. 22(1): p. 47-59.
32. Kennecke, H., et al., Metastatic behavior of breast cancer subtypes. J Clin Oncol, 2010. 28(20): p. 3271-7.
33. Wei, R., J. P. C. Wong, and H. F. Kwok, Osteopontin—a promising biomarker for cancer therapy. J Cancer, 2017. 8(12): p. 2173-2183.
34. Graudens, E., et al., Deciphering cellular states of innate tumor drug responses. Genome Biol, 2006. 7(3): p. R19.
35. Minn, A. J., et al., Genes that mediate breast cancer metastasis to lung. Nature, 2005. 436(7050): p. 518-24.
36. Richardson, A. L., et al., X chromosomal abnormalities in basal-like human breast cancer. Cancer Cell, 2006. 9(2): p. 121-32.
37. van de Vijver, M. J., et al., A gene-expression signature as a predictor of survival in breast cancer. N Engl J Med, 2002. 347(25): p. 1999-2009.
38. Ramankulov, A., et al., Elevated plasma osteopontin as marker for distant metastases and poor survival in patients with renal cell carcinoma. J Cancer Res Clin Oncol, 2007. 133(9): p. 643-52.
39. Shevde, L. A., et al., Osteopontin: an effector and an effect of tumor metastasis. Curr Mol Med, 2010. 10(1): p. 71-81.
40. Cook, A. C., et al., Osteopontin induces multiple changes in gene expression that reflect the six “hallmarks of cancer” in a model of breast cancer progression. Mol Carcinog, 2005. 43(4): p. 225-36.
41. Feng, F. and S. R. Rittling, Mammary tumor development in MMTV-c-myc/MMTV-v-Ha-ras transgenic mice is unaffected by osteopontin deficiency. Breast Cancer Res Treat, 2000. 63(1): p. 71-9.
42. Wai, P. Y., et al., Osteopontin silencing by small interfering RNA suppresses in vitro and in vivo CT26 murine colon adenocarcinoma metastasis. Carcinogenesis, 2005. 26(4): p. 741-51.
43. Wein, L. and S. Loi, Mechanisms of resistance of chemotherapy in early-stage triple negative breast cancer (TNBC). Breast, 2017. 34 Suppl 1: p. S27-S30.
44. Plumer, A., et al., Development of fragment-specific osteopontin antibodies and ELISA for quantification in human metastatic breast cancer. BMC Cancer, 2008. 8: p. 38.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of creating a plurality of barcoded clonal populations (BCPs), the method comprising:
(a) providing an initial heterogeneous population of cells;
(b) dividing the initial heterogeneous population of cells into separate cultures, each culture comprising a single cell from the initial heterogeneous population;
(c) maintaining the single cells in culture to provide a plurality of stable single cell-derived monoclonal populations; and
(d) introducing individual identifying nucleic acid sequences into each cell of the plurality of stable single cell-derived monoclonal populations; to thereby create a plurality of barcoded clonal populations (BCPs).

2. The method of claim 1, wherein the initial heterogeneous population of cells comprises cells from cancer cell lines or patient-derived cells, preferably cells comprising affected and/or normal cells.

3. The method of claim 1, wherein the identifying nucleic acid sequences comprise unique sequences of 10-40 nucleotides.

4. The method of claim 3, wherein the identifying nucleic acid sequences comprise unique sequences of 20-30 nucleotides, preferably 24 nucleotides.

5. The method of claim 1, wherein the identifying nucleic acid sequences are flanked by uniform sequences comprising PCR primer binding sites.

6. The method of claim 1, wherein the identifying nucleic acid sequences are integrated into the genomes of the cells of the plurality of stable single cell-derived monoclonal populations.

7. The method of claim 1 wherein the identifying nucleic acid sequences are introduced into the cells of the plurality of stable single cell-derived monoclonal populations using a viral vector.

8. The method of claim 5, wherein the viral vectors are lentiviral vectors.

9. The method of claim 1, further comprising mixing equal numbers of each BCP to create a barcoded polyclonal population of cells (BPP).

10. The method of claim 9, further comprising exposing the BPP to a test condition.

11. The method of claim 1, further comprising determining one or both of identity and relative abundance of each BCP in the BPP.

12. The method of claim 11, wherein identity and/or relative abundance of each BCP is determined using a method comprising PCR, a hybridization assay, or next-generation sequencing.

13. A method of selecting a therapy for a subject who has cancer, the method comprising: creating a plurality of barcoded clonal populations (BCPs) by a method comprising:
(a) providing an initial heterogeneous population of cells from the cancer in the subject;
(b) dividing the initial heterogeneous population of cells into separate cultures, each culture comprising a single cell from the initial heterogeneous population;
(c) maintaining the single cells in culture to provide a plurality of stable single cell-derived monoclonal populations; and
(d) introducing individual identifying nucleic acid sequences into each cell of the plurality of stable single cell-derived monoclonal populations; to thereby create a plurality of barcoded clonal populations (BCPs);
(e) mixing equal numbers of each BCP to create a barcoded polyclonal population of cells (BPP);
(f) exposing the BPP to a candidate therapeutic compound; and
determining one or both of identity and relative abundance of each BCP in the BPP.

14. The method of claim 13, wherein identity and/or relative abundance of each BCP is determined using a method comprising PCR, a hybridization assay, or next-generation sequencing.

15. The method of claim 13, wherein the initial heterogeneous population of cells comprises cells from cancer cell lines or patient-derived cells, preferably comprising affected and/or normal cells.

16. The method of claim 13, wherein the identifying nucleic acid sequences comprise unique sequences of 10-40 nucleotides.

17. The method of claim 16, wherein the identifying nucleic acid sequences comprise unique sequences of 20-30 nucleotides, preferably 24 nucleotides.

18. The method of claim 13, wherein the identifying nucleic acid sequences are flanked by uniform sequences comprising PCR primer binding sites.

19. The method of claim 13, wherein the identifying nucleic acid sequences are integrated into the genomes of the cells of the plurality of stable single cell-derived monoclonal populations.

20. The method of claim 13, wherein the identifying nucleic acid sequences are introduced into the cells of the plurality of stable single cell-derived monoclonal populations using a viral vector.

21. The method of claim 20, wherein the viral vectors are lentiviral vectors.

22. The method of claim 2, wherein the affected cells are tumor cells.
